(19) United States
(12) Patent Application Publication
(10) Pub. No.: US 2004/0208857 A1
(43) Pub. Date: Oct. 21, 2004

(54) Use of cells derived from embryonic stem cells for increasing transplantation tolerance and for repairing damaged tissue

(76) Inventors: Michael Bader, Berlin (DE); Bert Binas, Collage Station, TX (US); Giuxuan Chai, The Woodlands, CT (US); Fred Faendrich, Kiel (DE); Detlev Ganten, Neu-Buch (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/433,557

(22) PCT Filed: Dec. 4, 2001
(86) PCT No.: PCT/DE01/04512

(30) Foreign Application Priority Data
Dec. 4, 2000 (DE) 100 61 334.9

(51) Int. Cl. A61K 48/00; C12N 5/08; A01K 67/027

(52) U.S. Cl. 424/93.21; 435/366; 800/14

(57) ABSTRACT

The invention relates to the use of cells from cell lines, which are derived from early embryonic stages, for the donor-specific increase in transplantation tolerance and for repairing damaged tissue. Areas of application of the invention include the field of medicine and the pharmaceutical industry. The aim of the invention is to produce a donor-specific immunotolerance in order to prevent a rejection of the transplanted tissue due to an immune response and thus to be able to limit the administration of immune suppressive agents. In order to produce a donor-specific immunotolerance, embryonic stem cell-like cell lines (ECL) are obtained from blastocysts and are transfected with genetic material of the donor, which codes for the MHC haplotypes. The cells produced in such a manner are administered to the recipient before the transplantation for producing an immunotolerance against the tissue to be transplanted or for regenerating already damaged tissue.

The invention relates to the use of cells from cell lines derived from early embryonic stages, for donor-specific increase in transplantation tolerance and for repairing damaged tissue. Areas of application of the invention include the field of medicine and the pharmaceutical industry.

State of the Art

In transplantation medicine, the development of increasingly vigorous immune suppressive agents such as prednisone, cyclosporin, tacrolimus, mycophenolate mefetil, and anti-lymphocyte-antibody has increased the time of survival of the patients and the remaining time of the transplants by an average of one year. The routine use of these medicaments has rendered the clinical transplantation to become a standard treatment that is chosen for most of the non-malignant terminal disorders of the heart, the kidney and the liver.

An improvement of the duration of early survival of the transplants was, nevertheless, not achieved without a substantial infectious morbidity and non-immune side effects (Gaber et al., Transplantation 66: 29-37, 1998). In addition, a better duration of early survival could not be rendered into a better long-term duration of survival of the transplant, since the chronic further rejection of the transplants after the first year rendered them non functional with a frequency that has not essentially changed within the last 20 years (Cecka and Terasaki, Clinical Transplants 1997, Los Angeles, UCLA Tissue Typing Laboratory, 1998). Furthermore, during a longer follow-up of the clinical outcome of transplantations (Pirsch and Friedman, J. Gen. Intern. Med. 9: 29-37, 1994) showed an increasing late morbidity and mortality due to the further need of a non-specific immune suppression.

The term of immune tolerance (in the following designated as tolerance), which in general can be described as the absence of an immune reaction after the administration or uptake of a particular antigen (AG), plays a central role in transplantation medicine. From the point of view of a transplantation immunologist, the tolerance can be defined as the continuous persistence of a tissue in the absence of a harmful immune reaction that can be obtained without an ongoing therapeutical intervention. In this context, it is important to note that the tolerance is not a congenital characteristic of an individual but is acquired (Owen, Science 102: 400-401, 1945; Billingham et al., Nature 172: 603-606, 1953). It is furthermore known that the tolerant state that is present during birth is constantly changing and, in particular in cases, in which the body is confronted with new antigens during its entire life. The immune system must be able to tolerate, for example, certain “foreign” antigens, such as physiological hormones, released during puberty and pregnancy (Fowlkes and Ramsdell, Curr. Opin. Immunol. 5: 873-879, 1993). In addition, the fact that foetal life can develop and survive in a major histocompatibility complex (MRC) mismatched host, is another example for the ability of nature to be able to distinguish not only between foreign and non-foreign, but also between dangerous and non-dangerous (Vacchio and Jiang, Crit. Rev. Immunol. 19: 461-480, 1999).

In an allogeneic hematopoietic stem cell transplantation (CD34), a successful transplantation in an MHC mismatched host can only be achieved, if the acceptor is sublethally myeloablated or irradiated.

Disadvantages of the Current Transplantations of Organs and/or Tissue

In order to avoid a rejection of the transplant by an immune reaction, currently powerful immune suppressive agents are administered, which, in turn invoke an increased risk of infection. Thus, ubiquitous germs that do not represent a danger in a normally functioning immune system can cause severe diseases in an immune suppressive state. In a hematopoietic stem cell transplantation (CD34), for example, a successful transplantation in an MHC mismatched host can only be achieved if the bone marrow of the acceptor is previously removed or destroyed by X-ray irradiation.

### OBJECT OF THE INVENTION

It is an object of the present invention to generate donor-specific immune tolerance in order to prevent a rejection of the transplanted tissue by an immune reaction, and thus to limit the administration of immune suppressive agents.

### DESCRIPTION OF THE INVENTION

The invention is put into practice according to claim 1, the dependent claims are preferred embodiments.

For the generation of a donor-specific immune tolerance, embryonic stem-cell like cell lines (ECL) are obtained from blastocysts and transfected with genetic material of the donor that encodes for the MHC-haplotypes. The cells thus generated are finally administered to the acceptor before the transplantation for the generation of the immune tolerance against the tissue to be transplanted or for the renewal of already damaged tissue, respectively.

The use of cells from the ECLs as “tolerance vectors” is enabled by a lack of an MHC-antigen-expression and the immunogenic inactivity of the ECL related therewith. It was found during experiments that cells of ECLs can be transplanted and exhibit long-term survival whereby they generate hematopoietic cells of different origin. In addition, these ECL-derived hematopoietic cells derived from ECL generated a permanent mixed chimerism (hematopoietic cells of the donor and the recipient exist simultaneously in the same host) and therefore provide the basis for a long-term acceptance of the allo-transplant. Therefore, they can be either used as the ideal means for induction of tolerance or, alternatively, can be used in a situation, wherein the parenchymatic injury of a certain organ has to be remedied.

The invention is described in more detail in the following.

For ECL-isolation, three races of rats were chosen, Wistar Kyoto (WKY), Sprague Dawley (SD) and ACI.

Blastocysts obtained from these were seeded on mitomycin-inactivated embryo fibroblasts of mice (MEF) or rats (REF) as feeders. Die MEF proved to be better and both, MEF and REF, were clearly superior to gelatine. When the filaments of the inner cell mass (ICM) did not form after the attachment of blastocysts onto the feeder layer, they usually could be easily expanded in groups of ES-like cell clumps (primary clumps) for approximately 10 days (primary growth). After this, the cells largely differentiated into a mixture of different cell types and soon were overgrown by round, slightly attached, endoderm-like cells. Only a small portion of the cells from the cell clumps survived and were expanded to a cell line.

WKY-blastocysts attached very rapidly, showed a vigorous primary growth and more than 10% of the embryos yielded permanent cell lines (table 1). SD-blastocysts attached with a slight delay, yielded a moderate number of primary clumps and the effectiveness of the generation of cell lines was low. ACI-blastocysts required the longest time for the attachment, yielded a very small number of primary clumps and no cell line could be generated from this race. These findings propose that the velocity by which the blastocysts attach to the feeding cell layer, and the number of primary clumps during the largest primary growth are positively related with the effectiveness of the ECL-derivation (table 1). It is interesting that in case of the hybrid blastocysts, the WKY-genotype overrules the ACI in the production of ECLs but not the SD-genotype (table 1).

The use of the cells from the cell lines that were generated from embryonic stem cells as “tolerance vectors” for causing a donor-specific immune tolerance furthermore requires the expression of the donor-specific MHC-antigenes. This property is achieved according to the invention in that cells of the ECLs are transfected with the genes of the donor that encode for the MHC-antigenes. This can take place by (i) fusing the ECL with a given somatic cell or cell line that has the MHC-genes of interest, by (ii) transfection of the ECL with a given MHC-encoding plasmid, by (iii) generating transgenic rats with an MHC-encoding plasmid and the preparation of ECL from this race or by (iv) peptide-loading of the ECL with MHC-allopeptides of class I that encode for the highly polymorphic alpha 1-helix of a specific MHC-antigen of class I. For the administration of the transfected cells, several different variants, such as via the portal vein, by intraperitoneal, subcutaneous or intravenous injection are available.

In the clinical process of the transplantation of organs thereby the possibility is provided to modify the alloreaction of the recipient by the administration of ECLs that express the donor-specific MHC-antigenes. An exact phenotyping and morphological characterisation of the ECL-derived offspring allows for searching for similar cells having stem cell characteristics in the grown-up host. This enables one to arrive at a better understanding of the elasticity of the stem-cells derived from a fully grown tissue that, alternatively to the ECL, share the ECL-properties and can be used in a similar fashion.

### EXAMPLES

1. Isolation and Culturing of the Rat-ECL

Mouse embryo fibroblasts (MEF) or rat embryo fibroblasts (REF) were prepared from 13-14-days pregnant animals that were mitotically inactivated by 3-5 treatments with mitomycin C (10 mu g/ml) for 2 or 1 hours, washed with phosphate buffered saline (PBS) and seeded in Nunc 4-well-dishes. The blastocysts were flushed out with PBS/20% FCS (foetal calf serum) or a culture medium from the uterus of 4, 5 days pregnant rats, seeded on inactivated embryo fibroblasts and left untreated for 3-4 days in DMEM/15% FCS/2,500 mu/ml LIF (“Leukemia inhibiting factor”, ESGRO, Life Technologies) with supplements (Iannaccone et al., Dev. Biol. 1994; 163: 288-292) in a medium of 6% C02/air. During this time the blastocysts develop and attach to the feeder, and the ICM starts to grow, wherein the efficiency is depending from the genetic background. Filaments with an ES-cell like appearance are taken up and fractionated into several clumps by aspiration into drawn-out glass capillaries having a slightly smaller diameter than the filaments, and transferred onto fresh feeder plates. The taking-up and fractionating occurs either daily or on every second day. Disintegrated colonies were reset in series until one obtained a small number of clean, stably growing ES-like clumps. Die Population of the clumps is then expanded to several dozens, kept in 35-mm-dishes and slightly trypsinised in a mixture of single cells and small aggregates. Die produced rat-ES-cells were passaged each day or every second day by trypsinisation (0,05% trypsin, 0,02% EDTA-4Na, 1% chicken serum, in Ca/Mg-free PBS). The identity of the species of the resulting cell lines is checked by PCR using renin-gene-primers (Brenin et al., Transplant. Proc. 1997; 29: 1761-1765), in order to exclude a contamination by mouse ES-cells.

2. Intraportal Injection of WKY-derived ECL in Allogenic Rat-Hosts

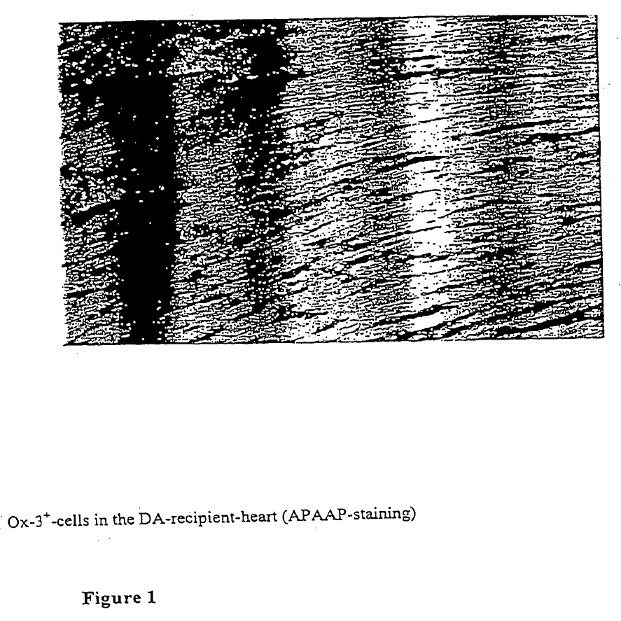

A first series of experiments investigated the fate of a single intraportal injection of 1,0×10 of WKY-derived ECL in allogenic DA(RT1.)-rat-hosts that did not receive any immune suppressive or myeloablative treatment. The experiments show that these cells have a long-term survival (>150 days) in DA-rat-hosts. During this, it was found that the ECL and their offspring are able to generate a status of a continuos permanent mixed chimerism (hematopoietic cells of the donor and the recipient co-exist in the same host). It was furthermore found that these cells differentiate into hematopoietic cells that express the MHC-antigenes of class II (Ox-3) and express B-cell derivation marker (Ox-45). The monoclonal antibody (mAb) Ox-3 is a specific antibody of a (WKY)-MHC-donor of class II that binds to MHC-epitopes of class II that are expressed on WKY, but not to positive DA MHC-cells of class II. A flow-cytometrical determination of double-stained leukocytes (WBC) resulted in that 3 to 5% of the WBC that were taken from DA-rats (100 days after the ECL-injection) expressed Ox-3 cells, wherein 15-20% of the population of spleen lymphocytes contained Ox-3. These results confirmed the fact that ECL can generate hematopoietic cells. Accordingly, Ox-3-cells were histomorphologically (10-15%) determined in the interstitial lumen of the recipient-(DA)-hearts, which were selectively destroyed 100 days after a unique intraportal injection of 1,0×10 of WKY derived ECL (see FIG. 1).

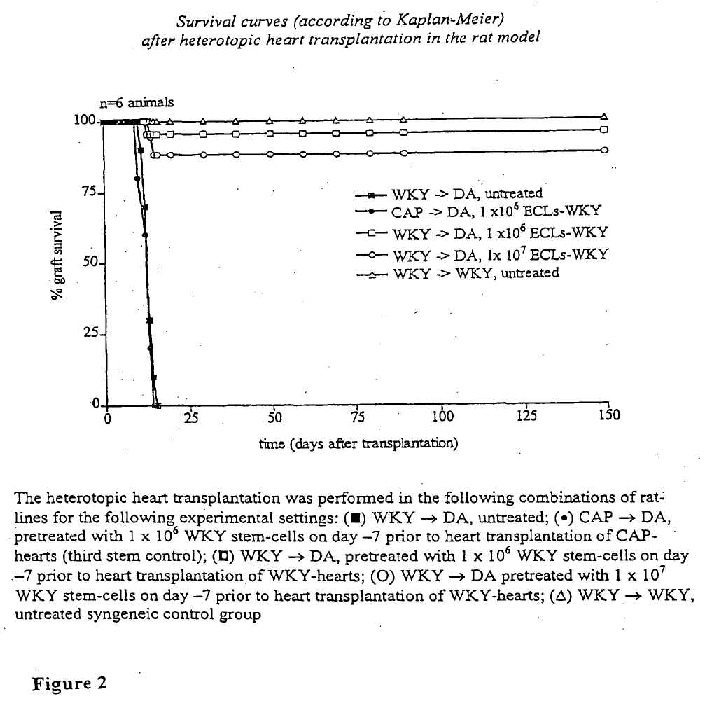

The stable chimerical status of these animals provides the basis for the examination of the fate of the WKY-heart allotransplants that were transplanted into DA-rats, seven days after the intraportal ECL-injection. Kaplan Meier diagrams show that the pre-treatment of the DA-rats with 1,0×10 ECL intraportal and the heart transplantation (HTx) seven days later led to a long-term (>150 days) rejection-free allotransplant acceptance, whereas non-treated DA-rats acutely rejected the WKY-allografts (see FIG. 2). Simultaneously the heart allotransplants of CAP-rats of DA-rats injected with WKY-ECL were rejected within 12.4+/−1.4 days, proving the immune competence of these rats.

3. Co-Cultivation of ECL with Somatic Cell Lines

It was shown in primary in vitro experiments that the ECL-cells as described before acquire a differentiation into astrocytes and cardiomyocytes and hepatocytes, respectively by co-cultivation with somatic cells of neuronal or entodermal origin. Thus, the embryonic cell lines as described are also suitable for the treatment of organ-specific diseases of the central nervous system (e.g. as dopamine cells for the treatment of Parkinson's disease, as hepatocytes for the treatment of liver cirrhosis or as cardiomyocytes for the treatment of recent heart attacks). The instant isolation of the signal protein required for these specific forms of differentiation is of great clinical relevance, since they could enable a smooth programming of the ECLs into the desired population of cells. Therefore, the goal consists in the exact sequencing of the functional proteins, in order to allow for their recombinant production. The great sequence homology between rat and human protein would in addition also give information for the analogous production of human functional proteins. The therapeutical uses connected therewith include both the above-described use of the ECL derived somatic cell lines and also functional proteins derived therefrom for the future clinical use at all levels of indications of the tissue-engineering for organ replacement, for gene therapy, and for the treatment of metabolic diseases in the area of the CNS, the liver and the heart.

1-18. (canceled)

19. A method for producing a donor-specific immunotolerance against transplanted tissue, comprising administering cells derived from an early stage of an embryo to a recipient of the transplanted tissue before transplantation.

20. The method as recited in claim 19 wherein the early stage of the embryo is a blastocyst.

21. The method as recited in claim 19 wherein the cells derived from an early stage of an embryo are derived from embryonic stem cell-like cell lines.

22. The method as recited in claim 19 further comprising, before the administering, transfecting the cells derived from an early stage of an embryo with at least one gene selected from the group consisting of MHC genes, reporter genes and combinations thereof.

23. The method as recited in claim 22 wherein the at least one gene is a donor-specific MHC gene.

24. The method as recited in claim 23 wherein the transfecting is performed by fusing the cells derived from an early stage of an embryo with cells expressing the donor-specific MHC gene.

25. The method as recited in claim 24 wherein the cells expressing the donor-specific MHC gene are selected from the group consisting of somatic cells expressing the donor-specific MHC gene and a cell line expressing the donor-specific MHC gene.

26. The method as recited in claim 23 wherein the transfecting is performed using a predetermined MHC coding plasmid.

27. The method as recited in claim 23 wherein the transfecting is performed by:
providing transgenic non-human mammals having a new MHC encoding plasmid; and
producing embryonic stem cell-like cell lines of the transgenic non-human mammals.

28. The method as recited in claim 23 wherein the cells derived from an early stage of an embryo are embryonic stem cell-like cell lines and wherein the transfecting is performed by peptide-loading the embryonic stem cell-like cell lines with MHC allopeptides of class I encoded for a highly polymorphic alpha 1 helix of a specific MHC antigen of class I.

29. The method as recited in claim 22 wherein the transfecting is performed using LacZ plasmid.

30. The method as recited in claim 19 wherein the cells derived from an early stage of an embryo include cells of a human cell line.

31. The method as recited in claim 19 wherein the administering is performed from three to seven days before the transplantation.

32. The method as recited in claim 19 wherein the administering is performed intravenously.

33. The method as recited in claim 19 wherein the administering is performed intraportally.

34. The method as recited in claim 19 wherein the administering is performed subcutaneously.

35. The method as recited in claim 19 wherein the administering is performed intraperitoneally.

36. The method as recited in claim 19 wherein the cells derived from an early stage of an embryo are derived from an embryonic stem cell-like cell line programmed as a starting cell for differentiation into neuronal cells having a predetermined transmitter function.

37. The method as recited in claim 36 wherein the transmitter function includes dopamine.

38. The method as recited in claim 19 wherein the cells derived from an early stage of an embryo are derived from an embryonic stem cell-like cell line programmed as a starting cell for differentiation into hepatocytes for supporting liver-specific metabolisms.

39. The method as recited in claim 19 wherein the cells derived from an early stage of an embryo are derived from an embryonic stem cell-like cell line programmed as a starting cell for differentiation into cardiomyocytes for regeneration of cardial muscle function.

40. The method as recited in claim 19 wherein signal proteins that exhibit a predetermined differentiation potential for at least one of neuronal dopamine-producing cells, hepatocytes and cardiomyocytes identified as a course of co-cultivation are produced in a form of recombinant proteins.
